(19) United States
(12) Patent Application Publication
(10) Pub. No.: US 2024/0166507 A1
(43) Pub. Date: May 23, 2024

(54) HYDROGEN-OXIDIZING BACTERIA CULTURING METHOD AND HYDROGEN-OXIDIZING BACTERIA CULTURING APPARATUS

(71) Applicant: SEIKO EPSON CORPORATION, TOKYO (JP)

(72) Inventors: Yasutoshi HIDESHIMA, Matsumoto-shi (JP); Naoko TOMITA, MATSUMOTO-SHI (JP); Fumiya MAEDA, MATSUMOTO-SHI (JP)

(21) Appl. No.: 18/486,618

(22) Filed: Oct. 13, 2023

(30) Foreign Application Priority Data
Nov. 17, 2022 (JP) 2022-183969

(51) Int. Cl. C01B 3/08; C12M 1/00; C12N 1/20

(52) U.S. Cl. CPC C01B 3/08; C12M 23/58; C12M 29/00; C12N 1/20

(57) ABSTRACT

A hydrogen-oxidizing bacteria culturing method includes: a hydrogen generating step of generating hydrogen by bringing a metal body into contact with a liquid containing water and causing a corrosion reaction in the metal body; and a hydrogen supplying step of supplying the generated hydrogen to a medium inoculated with hydrogen-oxidizing bacteria.

The present application is based on, and claims priority from JP Application Serial Number 2022-183969, filed Nov. 17, 2022, the disclosure of which is hereby incorporated by reference herein in its entirety.

### BACKGROUND

### 1. Technical Field

The present disclosure relates to a hydrogen-oxidizing bacteria culturing method and a hydrogen-oxidizing bacteria culturing apparatus.

### 2. Related Art

Hydrogen-oxidizing bacteria oxidize hydrogen into water to obtain energy, and perform carbon dioxide fixation to proliferate. Therefore, attempts have been made to produce various chemical products by culturing hydrogen-oxidizing bacteria using inorganic carbon such as carbon dioxide as a raw material. Accordingly, chemical products can be produced while contributing to the implementation of carbon neutrality.

WO 2019/207812 discloses, as a hydrogen-oxidizing bacteria culturing method, a method in which a medium is first placed in a culture vessel, a mixed gas containing hydrogen, oxygen, and carbon dioxide is next supplied into the culture vessel, and hydrogen-oxidizing bacteria are subjected to static culture or shaking culture in the culture vessel to which the mixed gas is supplied. WO 2019/207812 also discloses that, by optimizing a volume ratio among hydrogen, oxygen, and carbon dioxide in the mixed gas, the growth of the hydrogen-oxidizing bacteria gets better, and a target compound can be efficiently produced.

However, in the culture method described in WO 2019/207812, it is necessary to prepare a large amount of hydrogen, oxygen, and carbon dioxide in advance. In particular, since hydrogen is a combustible gas, it is necessary to pay close attention to the storage of hydrogen. Therefore, the culture method described in WO 2019/207812 has problems in that safety is necessary to be considered and the burden of equipment investment is large. In addition, in order to supply the mixed gas into the culture vessel, it is necessary to store the hydrogen gas or the mixed gas under pressurization or use a pump. Therefore, in consideration of social implementation of the hydrogen-oxidizing bacteria, it is required to reduce energy necessary for the storage and the supply of hydrogen.

Therefore, an object is to provide a mechanism by which hydrogen can be supplied without storing a large amount of hydrogen while reducing energy consumption in culture of hydrogen-oxidizing bacteria.

### SUMMARY

A hydrogen-oxidizing bacteria culturing method according to an application example of the present disclosure includes: a hydrogen generating step of generating hydrogen by bringing a metal body into contact with a liquid containing water and causing a corrosion reaction in the metal body; and a hydrogen supplying step of supplying the generated hydrogen to a medium inoculated with hydrogen-oxidizing bacteria.

A hydrogen-oxidizing bacteria culturing apparatus according to an application example of the present disclosure includes: a culture vessel; a metal body accommodated in the culture vessel and configured to generate hydrogen by causing a corrosion reaction by contact with a liquid containing water; and a medium accommodated in the culture vessel so as to be in contact with the generated hydrogen, and inoculated with hydrogen-oxidizing bacteria.

### BRIEF DESCRIPTION OF THE DRAWINGS

FIG. 1 is a schematic diagram showing a hydrogen-oxidizing bacteria culturing apparatus according to an embodiment.

FIG. 2 is a partial cross-sectional view showing an example of a metal body in FIG. 1.

FIG. 3 is a schematic diagram showing a hydrogen-oxidizing bacteria culturing apparatus according to a first modification of the embodiment.

FIG. 4 is a schematic diagram showing a hydrogen-oxidizing bacteria culturing apparatus according to a second modification of the embodiment.

FIG. 5 is a schematic diagram showing a hydrogen-oxidizing bacteria culturing apparatus according to a third modification of the embodiment.

FIG. 6 is a schematic diagram showing a hydrogen-oxidizing bacteria culturing apparatus according to a fourth modification of the embodiment.

FIG. 7 shows an example in which a permeation restricting portion shown in FIG. 6 is replaced with another member.

FIG. 8 shows an example in which the permeation restricting portion shown in FIG. 6 is replaced with another member.

FIG. 9 shows an example in which the permeation restricting portion shown in FIG. 6 is changed to another form.

FIG. 10 is a schematic diagram showing a hydrogen-oxidizing bacteria culturing apparatus according to a fifth modification of the embodiment.

FIG. 11 is a schematic diagram showing a hydrogen-oxidizing bacteria culturing apparatus according to a sixth modification of the embodiment.

FIG. 12 is a flowchart illustrating a hydrogen-oxidizing bacteria culturing method according to the embodiment.

### DESCRIPTION OF EMBODIMENTS

Hereinafter, a hydrogen-oxidizing bacteria culturing method and a hydrogen-oxidizing bacteria culturing apparatus according to the present disclosure will be described in detail based on embodiments shown in the accompanying drawings.

### 1. Hydrogen-Oxidizing Bacteria Culturing Apparatus

First, a hydrogen-oxidizing bacteria culturing apparatus according to an embodiment will be described.

As described above, the hydrogen-oxidizing bacteria are bacteria that grow using hydrogen as an energy source and can grow using carbon dioxide as a carbon source. A chemical product can be synthesized by using the hydrogen-oxidizing bacteria, hydrogen, and carbon dioxide. Accordingly, carbon dioxide, which is a greenhouse gas, can be used as a resource, and can contribute to the implementation of carbon neutrality.

In order to synthesize a chemical product in this manner, it is required to easily and efficiently culture the hydrogen-oxidizing bacteria. In particular, when a large amount of hydrogen gas is stored and transported, the time and effort and cost are necessary to meet various restrictions on safety. Therefore, it is required to reduce the amount of hydrogen gas stored and transported as much as possible. In addition, compression or liquefaction of a hydrogen gas is often required for the storage or transportation. A large amount of energy is consumed for the compression or liquefaction of the hydrogen gas, and therefore, a reduction in energy consumption is also a problem in culture of the hydrogen-oxidizing bacteria.

The present inventors have conducted intensive studies on methods for solving the above-described problems. Then, the present inventors have found a culturing apparatus that can generate hydrogen on demand in a place where the hydrogen-oxidizing bacteria are to be cultured and can feed the generated hydrogen to a medium with less energy consumption, and have completed the present disclosure.

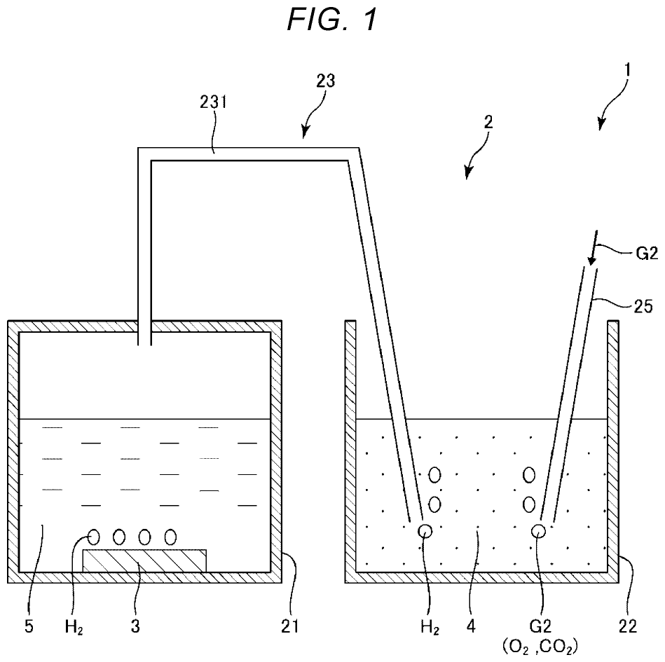

FIG. 1 is a schematic diagram showing a hydrogen-oxidizing bacteria culturing apparatus 1 according to the embodiment.

The hydrogen-oxidizing bacteria culturing apparatus 1 shown in FIG. 1 includes a culture vessel 2, a metal body 3, and a medium 4. The metal body 3 is accommodated in the culture vessel 2, and contact between the metal body 3 and a liquid containing water, that is, an aqueous solution 5 causes a corrosion reaction to generate hydrogen (H₂). The medium 4 is accommodated in the culture vessel 2 so as to be in contact with hydrogen generated by the corrosion reaction in the metal body 3. The medium 4 is inoculated with hydrogen-oxidizing bacteria.

According to the hydrogen-oxidizing bacteria culturing apparatus 1, hydrogen can be easily formed by the contact between the metal body 3 and the aqueous solution 5. Therefore, hydrogen can be supplied to the hydrogen-oxidizing bacteria inoculated in the medium 4. In this case, hydrogen can be supplied to the medium 4 without storing or transporting a large amount of hydrogen. In addition, it is not necessary to store hydrogen, and therefore, consumption of energy necessary for supplying hydrogen can be reduced. When only the term “hydrogen” is used in the present specification, the term “hydrogen” refers to a hydrogen molecule (H₂).

Hereinafter, each part of the hydrogen-oxidizing bacteria culturing apparatus 1 will be described in detail.

### 1.1. Culture Vessel

The culture vessel 2 shown in FIG. 1 includes a first accommodating unit 21, a second accommodating unit 22, a hydrogen transfer unit 23, and a gas supply unit 25.

### 1.1.1. First Accommodating Unit

The first accommodating unit 21 is a closed vessel that accommodates the metal body 3 and the aqueous solution 5. The aqueous solution 5 is stored in the first accommodating unit 21, and the metal body 3 is charged into the aqueous solution 5 at a timing when hydrogen is desired to be generated. Accordingly, hydrogen can be generated at a necessary timing without storing or transporting a large amount of hydrogen. The charging of the metal body 3 can be performed through, for example, an opening (not shown) provided in the first accommodating unit 21. The first accommodating unit 21 accommodating the metal body 3 may be replaced with a new one.

For the aqueous solution 5 and the medium 4, a water quality such as a temperature, pH, dissolved hydrogen, and dissolved oxygen may be monitored, and a gas component in an exhaust gas of the medium 4 may be monitored. The measurement results are fed back to appropriately adjust the water quality of the aqueous solution 5 such as a temperature and pH.

The aqueous solution 5 may be pure water, and is preferably water containing an electrolyte because pure water may have low conductivity. The electrolyte imparts good conductivity to the aqueous solution 5 and promotes the formation of a local battery.

Examples of the electrolyte include sodium chloride, hydrogen chloride, copper chloride, hydrogen sulfide, and sodium hydroxide. Therefore, for example, seawater is preferably used as the aqueous solution 5.

A concentration of the electrolyte in the aqueous solution 5 is not particularly limited, and is preferably 0.5 mass % or more, more preferably 1 mass % or more and 10 mass % or less, and still more preferably 2 mass % or more and 5 mass % or less. Accordingly, a reaction rate of corrosion can be particularly increased while reducing a consumption amount of the electrolyte. That is, even when the concentration of the electrolyte falls below the lower limit or exceeds the upper limit, the reaction rate may decrease depending on the temperature of the aqueous solution 5, the kind of the metal body 3, and the like.

The temperature of the aqueous solution 5 is not particularly limited, and is preferably 15° C. or higher, and more preferably 30° C. or higher. Accordingly, the reaction rate of corrosion can be further increased. The upper limit may not be particularly set, and is preferably set to 100° C. or less in consideration that the reaction rate of corrosion is too high or that handling is difficult.

A material of the first accommodating unit 21 is not particularly limited as long as it is a material that does not react with the aqueous solution 5 or does not degenerate due to hydrogen, and examples thereof include a metal material, a glass material, a ceramic material, and a resin material.

The first accommodating unit 21 shown in FIG. 1 is a closed vessel. Accordingly, the generated hydrogen can be transferred to the second accommodating unit 22 via the hydrogen transfer unit 23 with little leakage.

The first accommodating unit 21 may include a stirring device that stirs the aqueous solution 5, a shaking device that shakes the aqueous solution 5, and the like. Accordingly, the reaction rate of corrosion can be further increased.

Hydrogen generated on a surface of the metal body 3 tends to stay on the surface, and in order to prevent the tendency, the metal body 3 may be swung or subjected to ultrasonic vibration. The stirring, shaking, and ultrasonic vibration may be more appropriately adjusted based on the feedback of a monitoring result of the water quality of the aqueous solution 5 and the medium 4 such as a temperature and pH or a monitoring result of an exhaust gas component of the medium 4.

### 1.1.2. Second Accommodating Unit

The second accommodating unit 22 is a vessel that accommodates the medium 4. In FIG. 1, the second accommodating unit 22 is an open vessel, or may be a closed vessel. The medium 4 shown in FIG. 1 is in a liquid state. The hydrogen-oxidizing bacteria to be cultured are inoculated in the medium 4.

The hydrogen transferred through the hydrogen transfer unit 23 is released in the medium 4. Accordingly, the hydrogen is bubbled and the bubbled hydrogen is supplied to the medium 4.

A tip of the hydrogen transfer unit 23 immersed in the medium 4 has a simple shape in FIG. 1, or may have a doughnut shape, a spiral shape, a radial shape, or another complicated shape, or may have countless holes.

On the other hand, carbon dioxide or oxygen and carbon dioxide are supplied to the medium 4 from the gas supply unit 25. The hydrogen-oxidizing bacteria are often facultative anaerobic or microaerophilic, and therefore, oxygen is preferably supplied to the medium 4. Carbon dioxide is a carbon source necessary for production of chemical products.

A material of the second accommodating unit 22 is not particularly limited as long as it is a material that does not react with the medium 4 or does not degenerate due to hydrogen, and examples thereof include a metal material, a glass material, a ceramic material, and a resin material.

The second accommodating unit 22 may include a stirring device that stirs the medium 4, a shaking device that shakes the medium 4, and the like. Accordingly, a culture rate of the hydrogen-oxidizing bacteria can be further increased.

The temperature, pH, dissolved oxygen concentration, dissolved hydrogen concentration, or the like of the medium 4 may be monitored, and an exhaust gas component in the medium 4 may be monitored. The measurement results are fed back to appropriately adjust the temperature, pH, dissolved oxygen concentration, dissolved hydrogen concentration, or the like.

The gas not used in the reaction may be recycled again and supplied to the medium 4.

### 1.1.3. Hydrogen Transfer Unit

The hydrogen transfer unit 23 includes a pipe 231 that connects the first accommodating unit 21 and the second accommodating unit 22 at a position thereabove. One end of the pipe 231 is airtightly coupled to an upper portion of the first accommodating unit 21, and the other end of the pipe 231 is open in the medium 4 accommodated in the second accommodating unit 22.

A film such as a hydrogen permeable film or a filter (not shown) may be used for the hydrogen transfer unit 23. Accordingly, hydrogen can be supplied more selectively.

A valve or a pump (not shown) may be used. Also in this case, the pressure of hydrogen in the first accommodating unit 21 can be used, and therefore, a capacity of the pump can be reduced. Accordingly, energy consumption in the supply of hydrogen can be reduced.

The hydrogen generated in the first accommodating unit 21 rises due to a buoyant force and moves from one end toward the other end of the pipe 231. Accordingly, the hydrogen can be transferred from the first accommodating unit 21 to the second accommodating unit 22. Since a closed vessel is used as the first accommodating unit 21, hydrogen having a pressure higher than the atmospheric pressure can be generated without performing an operation of compressing hydrogen. Therefore, the hydrogen transfer unit 23 can bubble the hydrogen in the medium 4 without using a pump. Accordingly, the hydrogen can be supplied to the medium 4 while reducing energy consumption. Necessary hydrogen is also changed in the process of culturing the hydrogen-oxidizing bacteria, so that the water quality such as a temperature, pH, dissolved oxygen concentration, and dissolved hydrogen concentration of the medium 4 may be monitored, the measurement results are fed back, and an amount of hydrogen to be bubbled may be adjusted by an adjustment device such as a valve or a pump provided in the pipe 231. When an amount of hydrogen generated is insufficient, an amount of the metal body 3 in the aqueous solution 5 can be increased, the water quality such as a temperature and pH can be appropriately adjusted, a swing amount of the metal body 3 can be increased, or ultrasonic vibration can be increased. The culture vessel 2 can be divided into the first accommodating unit 21 and the second accommodating unit 22 by using the hydrogen transfer unit 23. Accordingly, the aqueous solution 5 and the medium 4 can be stored separately, and therefore, for example, the influence of the components in the aqueous solution 5 on the growth of the hydrogen-oxidizing bacteria can be avoided.

### 1.1.4. Gas Supply Unit

The gas supply unit 25 supplies carbon dioxide or a gas G2 containing oxygen and carbon dioxide to the medium 4 accommodated in the second accommodating unit 22. Accordingly, carbon dioxide or oxygen and carbon dioxide can be continuously supplied to the medium 4. As a result, the hydrogen-oxidizing bacteria can be continuously cultured, and production of chemical products based on carbon dioxide fixation can be continuously performed. The gas G2 may contain components other than oxygen and carbon dioxide. The gas supplied from the gas supply unit 25 may be appropriately adjusted depending on the kind of the hydrogen-oxidizing bacteria to be cultured, and further, the supplied gas may be appropriately adjusted by monitoring the water quality such as a temperature, pH, dissolved oxygen concentration, and dissolved hydrogen concentration, and feeding back the measurement results. The gas containing carbon dioxide is, for example, a combustion gas or the like generated by combustion, and can be supplied as it is when there is no or little influence on the culture of the hydrogen-oxidizing bacteria. Further, before being supplied to the medium 4, the combustion gas or the like may be refined to remove a component that influences the culture of the hydrogen-oxidizing bacteria.

The oxygen supplied by the gas supply unit 25 functions as an electron acceptor in, for example, the culture of specific hydrogen-oxidizing bacteria, and is used for the purpose of oxidizing hydrogen. The carbon dioxide supplied by the gas supply unit 25 is fixed in the hydrogen-oxidizing bacteria, and is used as a raw material of a chemical product.

As for the volume ratio of each gas necessary for culturing the hydrogen-oxidizing bacteria, a ratio of hydrogen:oxygen:carbon dioxide is about 8:1:1 as an example. The volume ratio may be out of this ratio, and basically, the volume ratio of hydrogen is the largest. Therefore, a flow rate of the gas G2 may be sufficiently smaller than that of hydrogen. Therefore, the energy necessary for an operation of the gas supply unit 25 is also sufficiently reduced.

### 1.2. Metal Body

Examples of a form of the metal body 3 include a powder form, a granule form, a block form, a chip form, a plate form, a rod form, and a linear form. A molded body having a more complicated shape may be formed by molding. A specific surface area changes depending on the form of the metal body 3. The specific surface area of the metal body 3 influences a generation rate of hydrogen, and therefore, the form of the metal body 3 may be selected according to the desired generation rate of hydrogen.

A material of the metal body 3 is not particularly limited as long as it can be corroded by the contact with the aqueous solution 5 to generate hydrogen. The metal body 3 particularly preferably contains a metal element having an ionization tendency higher than that of hydrogen. Accordingly, the metal body 3 efficiently generates hydrogen by the contact with the aqueous solution 5.

Examples of the metal element having an ionization tendency higher than that of hydrogen include Li, K, Ca, Na, Mg, Al, Ti, Zn, Fe, Co, Ni, Sn, and Pb. Among them, Ca, Mg, Al, Ti, or Zn is preferably used in consideration of handleability, hydrogen generation efficiency, and the like.

As the material of the metal body 3, a metallic element or a compound containing these metal elements is preferably used, and in particular, a metallic element or a hydrogenated compound including these metal elements is more preferably used. When the material is a metallic element or a hydrogenated compound, the metal body 3 having particularly good reactivity with the aqueous solution 5 can be implemented.

Specific examples of the material of the metal body 3 include elemental calcium, a calcium-based alloy, calcium hydride, elemental magnesium, a magnesium-based alloy, magnesium hydride, elemental aluminum, an aluminum-based alloy, aluminum hydride, or composite materials containing them.

Among them, elemental magnesium, a magnesium-based alloy, magnesium hydride, or a composite material containing any one of them is preferably used, and a magnesium-based alloy or a composite material containing a magnesium-based alloy is more preferably used. They are useful as the material of the metal body 3 because the hydrogen generation efficiency is particularly high.

Examples of the magnesium-based alloy include AZ91A, AZ91B, AZ91D, AM60A, AM60B, AS41A, AZ31, AZ31B, AZ61A, AZ63A, AZ80A, AZ91C, AZ91E, AZ92A, AM100A, ZK51A, ZK60A, ZK61A, EZ33A, QE22A, ZE41A, M1A, WE54A, and WE43B of standards of the American Society for Testing and Materials (ASTM).

On the other hand, examples of the composite material include a composite material of a material containing the above-described metal element and a material containing a component that promotes corrosion. The former material constitutes, for example, a matrix portion of the metal body 3, and the latter material constitutes particles dispersed in the matrix portion. With such a composite material, when the metal body 3 comes into contact with the aqueous solution 5, a local battery is formed between the matrix portion and the particles, and the local battery is formed evenly over the entire metal body 3. Accordingly, a corrosion reaction can be caused at a high rate, and a decrease in reaction rate due to a by-product is easily prevented.

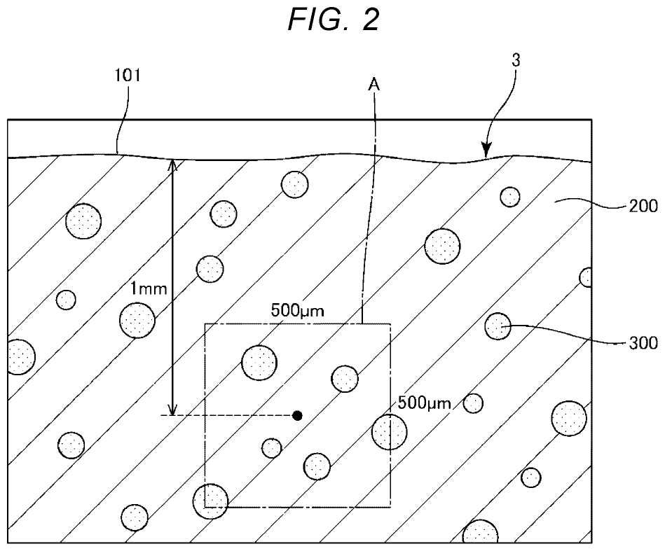

FIG. 2 is a partial cross-sectional view showing an example of the metal body 3 in FIG. 1. The metal body 3 shown in FIG. 2 includes a matrix portion 200, and a particle portion 300 dispersed in the matrix portion 200. The shapes and distribution state of the particle portion 300 shown in FIG. 2 are schematic.

The matrix portion 200 is made of a material containing the above-described metal elements, and is a portion mainly to be corroded by a corrosion reaction. The matrix portion 200 occupies a larger area fraction than the particle portion 300 in a cross section of the metal body 3.

The particle portion 300 is made of a material containing, as a main component, the above-described component which promotes corrosion (corrosion promoting component). The corrosion promoting component causes galvanic corrosion due to a potential difference with the matrix portion 200. Accordingly, the particle portion 300 promotes corrosion of the matrix portion 200. The corrosion promoting component is preferably any one of Fe, Ni, Co, Cu, carbon, and compounds containing at least one of them. They particularly promote the corrosion of the matrix portion 200 because they are components having particularly low cathode overvoltage. The corrosion promoting component being the main component in the particle portion 300 can be identified based on that an element content of any one of Fe, Ni, Co, Cu, and carbon in terms of an atomic ratio is the highest as a result of an elemental analysis. For the elemental analysis, for example, a qualitative and quantitative analysis based on an energy dispersive X-ray spectroscopy (EDX) is used. The element content of Fe, Ni, Co, or Cu in the particle portion 300 may be higher than that of other elements, and is preferably more than 20 atomic %, and more preferably more than 40 atomic %. During the identification of the particle portion 300 in the qualitative and quantitative analysis, the particle portion 300 can be distinguished based on a contrast from other portions or a color tone in, for example, an observation image from a scanning electron microscope or an optical microscope. The particle portion 300 may contain an additive or an impurity other than the corrosion promoting component.

Specific examples of the corrosion promoting component include elemental iron, iron-based compounds such as an iron oxide, an iron carbide, an iron nitride, an iron chloride, an iron sulfide, an iron carbonate, and an iron hydroxide, elemental nickel, nickel compounds such as a nickel oxide, a nickel carbide, a nickel nitride, a nickel chloride, a nickel sulfide, a nickel carbonate, and a nickel hydroxide, elemental cobalt, cobalt compounds such as a cobalt oxide, a cobalt carbide, a cobalt nitride, a cobalt chloride, a cobalt sulfide, a cobalt carbonate, and a cobalt hydroxide, elemental copper, copper compounds such as a copper oxide, a copper carbide, a copper nitride, a copper chloride, a copper sulfide, a copper carbonate, and copper hydroxide, and carbon such as graphite, carbon black, and carbon fibers.

Among them, the corrosion promoting component preferably contains elemental Cu or a Cu-based compound as a main component. The Cu-based compound is particularly preferably a copper oxide, a Cu—Al-based compound, or a Cu—Al—Mg-based compound. Accordingly, the hydrogen generation efficiency of the metal body 3 can be particularly increased. Even when a magnesium oxide layer is formed in the matrix portion 200, the efficiency of breaking the layer is considered to be high, and therefore, the metal body 3 that can generate hydrogen for a longer period of time can be implemented.

Examples of the copper oxide include CuO and Cu₂O. The Cu—Al-based compound is a compound in which a content of Cu is the highest and a content of Al is the second highest in terms of atomic ratio. The Cu—Al—Mg-based compound is a compound in which contents of Cu, Al, and Mg are from the highest to the lowest in this order in terms of atomic ratio.

An abundance ratio between the matrix portion 200 and the particle portion 300 is determined based on an area ratio in an observation image of a cross section of the metal body 3.

In the observation image of the cross section of the metal body 3 shown in FIG. 2, a range A of 500 μm square is set around a point at a depth of 1 mm from a surface 101. A proportion of an area of the particle portion 300 to an area of the range A is defined as an area fraction of the particle portion 300.

The area fraction is preferably 0.5% or more and 20.0% or less, more preferably 1.08 or more and 15.0% or less, and still more preferably 2.0% or more and 10.0% or less. By setting the area fraction of the particle portion 300 within the above range, quantitative balance between the matrix portion 200 and the particle portion 300 is optimized. Therefore, the metal body 3 in which the particle portion 300 is uniformly distributed is obtained without impairing mechanical strength of the metal body 3. In such a metal body 3, the corrosion promoting component is uniformly dispersed, and therefore, the local battery formed between the corrosion promoting component and the matrix portion 200 is distributed without bias. Therefore, even if a by-product inhibiting hydrogen generation is generated, the entire surface of the metal body 3 is hardly covered with the by-product, and as a result, corrosion can be continued. Therefore, according to the metal body 3 as shown in FIG. 2, the probability of continuously and efficiently generating hydrogen until the matrix portion 200 is lost increases.

When the area fraction falls below the lower limit, the particle portion 300 is insufficient, and therefore, depending on the materials of the matrix portion 200 and the particle portion 300, the generation of hydrogen may be inhibited by the by-product. On the other hand, when the area fraction exceeds the upper limit, the particle portion 300 is excessive, and therefore, the mechanical strength of the metal body 3 is decreased and the ratio of the matrix portion 200 is relatively decreased, leading to a decrease in hydrogen generation efficiency.

The area fraction in the range A is calculated as follows. First, a range of the particle portion 300 is extracted by image processing in the range A. For the image processing, for example, image analysis software OLYMPUS Stream or the like can be used. A magnification of the observation image is preferably 300 times or more. Next, a proportion of the area of the particle portion 300 to the entire area of the range A is calculated. The proportion is defined as the area fraction.

An average particle diameter of the particle portion 300 is preferably 30.0 μm or less, more preferably 0.2 μm or more and 15.0 μm or less, and still more preferably 0.5 μm or more and 10.0 μm or less. Accordingly, the particle portion 300 is less likely to become a starting point of breakage, and therefore, a decrease in mechanical strength of the metal body 3 can be prevented. The particle portion 300 can be more uniformly distributed, and therefore, hydrogen generation inhibition caused by the by-product is less likely to occur.

The average particle diameter of the particle portion 300 is calculated as follows. First, a length of a major axis and a length of a minor axis of the particle portion 300 included in the range A are determined. Next, an intermediate value between the length of the major axis and the length of the minor axis is determined. An average value of the intermediate values calculated in this manner is the average particle diameter of the particle portion 300.

An average aspect ratio of the particle portion 300 is preferably 4.0 or less, more preferably 3.0 or less, and still more preferably 2.0 or less. When the average aspect ratio of the particle portion 300 is within the above range, anisotropy of the structure of the particle portion 300 is reduced. Therefore, the mechanical strength and rigidity of the metal body 3 can be increased isotropically. Accordingly, the impact resistance of the metal body 3 can be improved.

The average aspect ratio of the particle portion 300 is calculated as follows. First, a length of a major axis and a length of a minor axis of the particle portion 300 included in the range A are determined. Next, a ratio of the length of the major axis to the length of the minor axis is referred to as an “aspect ratio”. An average value of the aspect ratios calculated in this manner is the average aspect ratio of the particle portion 300.

### 1.3. Medium

The medium 4 is, for example, a liquid medium obtained by dispersing an organic medium or an inorganic medium in water. The organic medium is, for example, a medium containing organic substances such as sugars, organic acids, and amino acids. Examples of the inorganic medium include a medium containing carbonates. Hydrogen-oxidizing bacteria are inoculated in the medium 4. The hydrogen-oxidizing bacteria grow by hydrogen and oxygen supplied to the medium 4, and implement carbon dioxide fixation. As a result, various chemical products are produced.

The hydrogen-oxidizing bacteria are not particularly limited as long as they can grow using hydrogen as an energy source and carbon dioxide as a carbon source as described above. Specific examples of the hydrogen-oxidizing bacteria include a transformant of Hydrogenophilus bacteria described in WO 2019/207812.

The temperature of the medium 4 is appropriately set according to the kind of the hydrogen-oxidizing bacteria, and is preferably 30° C. or higher and 60° C. or lower, and more preferably 35° C. or higher and 55° C. or lower as an example.

The pH of the medium 4 is not particularly limited, and is preferably 6.2 or more and 8.0 or less, and more preferably 6.4 or more and 7.5 or less. Accordingly, the growth of the hydrogen-oxidizing bacteria gets better, and the chemical products can be produced more efficiently.

### 2. Modifications

Next, hydrogen-oxidizing bacteria culturing apparatuses according to modifications of the embodiment will be described.

### 2.1. First Modification

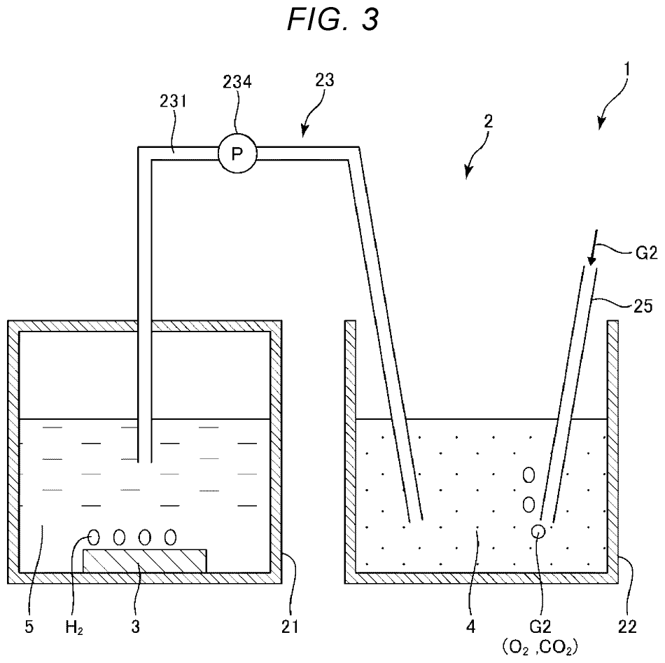

FIG. 3 is a schematic diagram showing the hydrogen-oxidizing bacteria culturing apparatus 1 according to a first modification of the embodiment.

Hereinafter, the first modification will be described, and in the following description, differences from the embodiment will be mainly described, and description of similar matters will be omitted. In FIG. 3, the same components as those in the embodiment are denoted by the same reference signs.

The hydrogen-oxidizing bacteria culturing apparatus 1 shown in FIG. 3 is the same as the hydrogen-oxidizing bacteria culturing apparatus 1 shown in FIG. 1 except that a configuration of the hydrogen transfer unit 23 is different.

The hydrogen transfer unit 23 shown in FIG. 3 includes the pipe 231 and a pump 234 provided in the middle of the pipe 231. One end of the pipe 231 is open in the aqueous solution 5. The hydrogen transfer unit 23 including the pump 234 transfers the aqueous solution 5 containing dissolved hydrogen to the second accommodating unit 22. In the aqueous solution 5, hydrogen generated from the metal body 3 is dissolved in a saturated state or a state close to the saturated state. That is, dissolved hydrogen having a high concentration can be efficiently transferred by transferring the aqueous solution 5 containing the dissolved hydrogen instead of hydrogen as a gas. As a result, the hydrogen-oxidizing bacteria can be efficiently cultured in the medium 4.

### 2.2. Second Modification

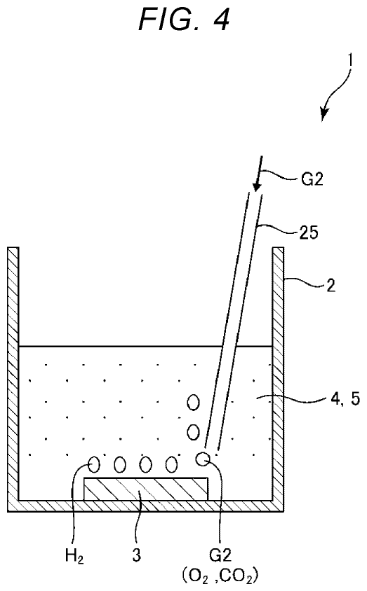

FIG. 4 is a schematic diagram showing the hydrogen-oxidizing bacteria culturing apparatus 1 according to a second modification of the embodiment.

Hereinafter, the second modification will be described, and in the following description, differences from the above-described embodiment will be mainly described, and description of similar matters will be omitted. In FIG. 4, the same components as those in the embodiment are denoted by the same reference signs.

The hydrogen-oxidizing bacteria culturing apparatus 1 shown in FIG. 4 is the same as the hydrogen-oxidizing bacteria culturing apparatus 1 shown in FIG. 1 except that the culture vessel 2 accommodates the aqueous solution 5 and the medium 4 in a mixed state. That is, the culture vessel 2 includes the first accommodating unit 21 and the second accommodating unit 22 in the hydrogen-oxidizing bacteria culturing apparatus 1 shown in FIG. 1, whereas the hydrogen-oxidizing bacteria culturing apparatus 1 shown in FIG. 4 includes one culture vessel 2 in which the first accommodating unit 21 and the second accommodating unit 22 described above are integrated. In such a culture vessel 2, generation of hydrogen from the metal body 3 and supply of hydrogen to the medium 4 are simultaneously performed. That is, the aqueous solution 5 and the medium 4 are mixed, and therefore, the time and effort for supplying the generated hydrogen can be saved. Such a culture vessel 2 has a simpler structure and can reduce consumption of energy necessary for supplying hydrogen.

According to such a second modification, hydrogen can be efficiently generated and supplied to the medium 4 only by charging the metal body 3 and supplying a small amount of the gas G2 while simplifying the structure of the culture vessel 2.

### 2.3. Third Modification

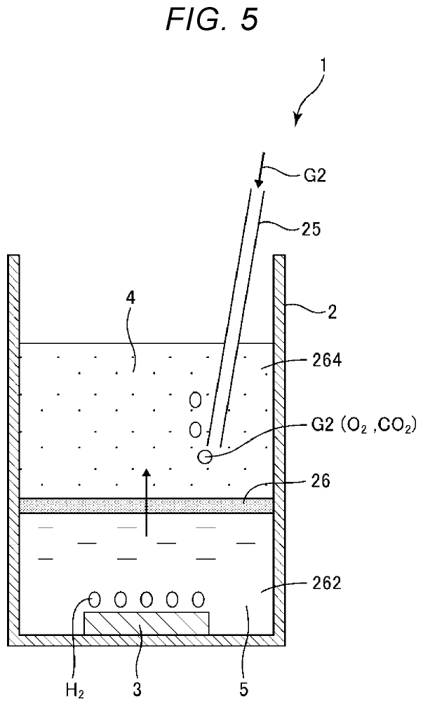

FIG. 5 is a schematic diagram showing the hydrogen-oxidizing bacteria culturing apparatus 1 according to a third modification of the embodiment.

Hereinafter, the third modification will be described, and in the following description, differences from the embodiment will be mainly described, and description of similar matters will be omitted. In FIG. 5, the same components as those in the embodiment are denoted by the same reference signs.

The hydrogen-oxidizing bacteria culturing apparatus 1 shown in FIG. 5 is the same as the hydrogen-oxidizing bacteria culturing apparatus 1 shown in FIG. 4 except that a gas permeable film 26 that partitions an interior of the culture vessel 2 into upper and lower portions is provided.

In the interior of the culture vessel 2, a portion below the gas permeable film 26 is referred to as a lower portion 262, and a portion above the gas permeable film 26 is referred to as an upper portion 264. The metal body 3 and the aqueous solution 5 are accommodated in the lower portion 262. The medium 4 inoculated with hydrogen-oxidizing bacteria is accommodated in the upper portion 264.

The culture vessel 2 shown in FIG. 5 includes the gas supply unit 25. The gas supply unit 25 supplies the gas G2 to the upper portion 264.

In such a hydrogen-oxidizing bacteria culturing apparatus 1, the aqueous solution 5 and the medium 4 can be separated from each other while using one culture vessel 2. Accordingly, the generation of hydrogen and the growth of the hydrogen-oxidizing bacteria can be performed in spaces independent of each other. As a result, the hydrogen generation efficiency and the culture efficiency of the hydrogen-oxidizing bacteria can be further increased while simplifying the structure of the culture vessel 2.

The gas permeable film 26 selectively allows hydrogen to permeate, and therefore, the transfer of the by-product and the like accompanying the generation of hydrogen to the medium 4 can be prevented.

### 2.4. Fourth Modification

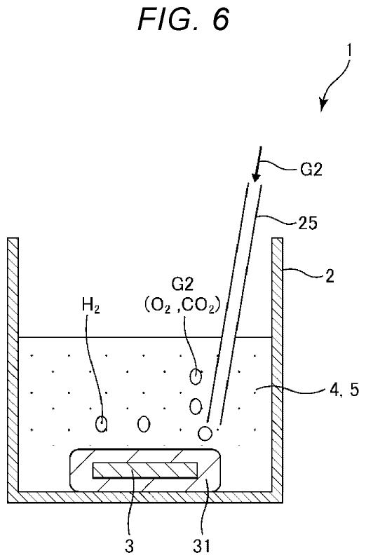

FIG. 6 is a schematic diagram showing the hydrogen-oxidizing bacteria culturing apparatus 1 according to a fourth modification of the embodiment.

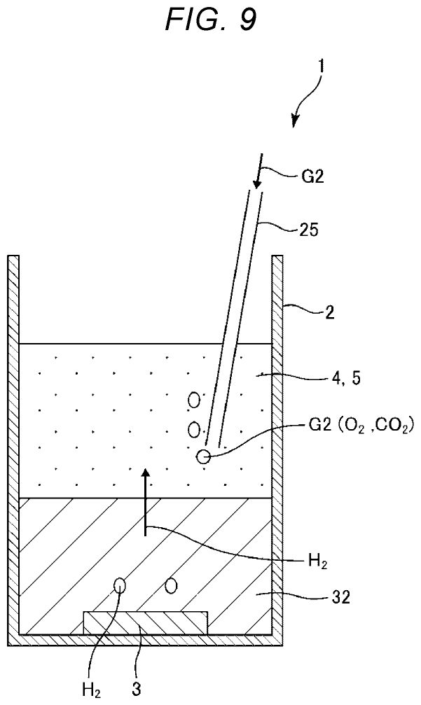

Hereinafter, the fourth modification will be described, and in the following description, differences from the embodiment will be mainly described, and description of similar matters will be omitted. In FIGS. 6 and 9, the same components as those in the embodiment are denoted by the same reference signs.

The hydrogen-oxidizing bacteria culturing apparatus 1 shown in FIG. 6 is the same as the hydrogen-oxidizing bacteria culturing apparatus 1 shown in FIG. 4 except that the hydrogen-oxidizing bacteria culturing apparatus 1 shown in FIG. 6 includes a permeation restricting portion 31 that covers a periphery of the metal body 3.

The permeation restricting portion 31 shown in FIG. 6 has a gel shape covering a surface of the metal body 3. The permeation restricting portion 31 has a function of restricting permeation rates of hydrogen and the aqueous solution 5. Therefore, by providing the permeation restricting portion 31, the chance of contact between the metal body 3 and the aqueous solution 5 is reduced, and the reaction rate of corrosion can be decreased. When hydrogen generated by corrosion rises, the rising speed is reduced by the permeation restricting portion 31. With these actions, the amount of hydrogen generated can be adjusted. The function of restricting the permeation rate is a function of reducing both the permeation rate of hydrogen and the permeation rate of the aqueous solution 5 compared with a case where the permeation restricting portion 31 is not provided.

When the metal body 3 is covered with the gel substance, a corrosion product of the metal body 3 can be prevented from coming into direct contact with the hydrogen-oxidizing bacteria. Therefore, there is also an advantage that the corrosion product does not influence the culture of the hydrogen-oxidizing bacteria. The dissolved hydrogen concentration in the medium 4 has an upper limit, and even if a large amount of hydrogen is supplied at one time, the hydrogen cannot be supplied to the medium 4, which is wasted. In such a case, when the generation rate of hydrogen can be reduced, an amount of hydrogen to be wasted can be reduced, and the corrosion of the metal body 3 can be maintained over a longer period of time. Accordingly, the consumption efficiency of the metal body 3 can be increased.

The generation rate of hydrogen can be adjusted according to the thickness and density of the permeation restricting portion 31. Further, the permeation restricting portion 31 can be integrally handled while being attached to the metal body 3.

Examples of a material of the permeation restricting portion 31 include various elastomer gels. Examples of the elastomer gel include: plant-based polymers such as gum arabic, carrageenan, and agar; microorganism-based polymers such as xanthan gum and dextran; animal-based polymers such as collagen and gelatin; starch-based polymers such as carboxymethyl starch; cellulose-based polymers such as methyl cellulose; alginic acid-based polymers such as sodium arginate; vinyl-based polymers such as polyvinyl methyl ether; polyoxyethylene-based polymers; polyoxyethylene-polyoxypropylene copolymer-based polymers; acrylic polymers such as sodium polyacrylate; synthetic water-soluble polymers such as polyethyleneimine; and inorganic water-soluble polymers such as bentonite, aluminum magnesium silicate, montmorillonite, beidellite, nontronite, saponite, hectorite, and silicic anhydride.

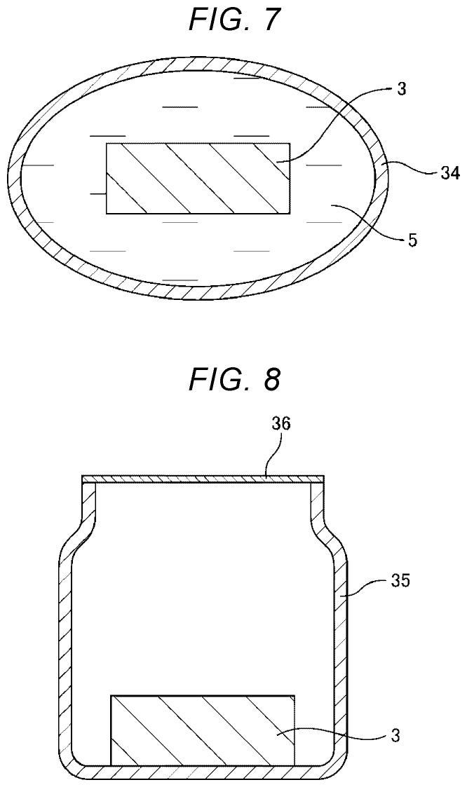

FIGS. 7 and 8 show an example in which the permeation restricting portion 31 shown in FIG. 6 is replaced with another member.

FIG. 7 is a schematic diagram showing a permeation restricting portion 34 covering the periphery of the metal body 3.

The permeation restricting portion 31 described above may be replaced with the permeation restricting portion 34 shown in FIG. 7. Similar to the permeation restricting portion 31, the permeation restricting portion 34 has a function of restricting the permeation rates of hydrogen and the aqueous solution 5. The permeation restricting portion 34 may have a closed film shape, and an interior thereof may be filled with the aqueous solution 5 in advance. The metal body 3 is charged into the culture vessel 2 shown in FIG. 6 with such a permeation restricting portion 34. In such a case, the same effect as that of the hydrogen-oxidizing bacteria culturing apparatus 1 shown in FIG. 6 can also be obtained.

FIG. 8 is a schematic diagram showing a vessel 35 that accommodates the metal body 3 therein and has an opening, and a permeation restricting portion 36 that closes the opening.

The permeation restricting portion 31 described above may be replaced with the vessel 35 and the permeation restricting portion 36 shown in FIG. 8. When the vessel 35 accommodates the metal body 3, contact between the metal body 3 and the aqueous solution 5 is restricted, and storage and handling of the metal body 3 are facilitated. That is, even when the vessel 35 is left in a high-temperature and high-humidity environment, the metal body 3 can be prevented from degenerating. Therefore, a material having liquid tightness or airtightness is used as a material of the vessel 35. Similar to the permeation restricting portion 31, the permeation restricting portion 36 has a function of restricting the permeation rates of hydrogen and the aqueous solution 5. The metal body 3 is charged into the culture vessel 2 shown in FIG. 6 with the vessel 35 and the permeation restricting portion 36. In such a case, the same effect as that of the hydrogen-oxidizing bacteria culturing apparatus 1 shown in FIG. 6 can also be obtained.

By providing the permeation restricting portions 34 and 36 as described above, the chance of the contact between the metal body 3 and the aqueous solution 5 is reduced, and the reaction rate of corrosion can be decreased. When hydrogen generated by corrosion rises, the rising speed is reduced by the permeation restricting portions 34 and 36. With these actions, the amount of hydrogen generated can be adjusted.

FIG. 9 shows an example in which the permeation restricting portion 31 shown in FIG. 6 is changed to another form.

The hydrogen-oxidizing bacteria culturing apparatus 1 shown in FIG. 9 is the same as the hydrogen-oxidizing bacteria culturing apparatus 1 shown in FIG. 4 except that the lower portion of the culture vessel 2 is filled with a permeation restricting portion 32.

The lower portion of the culture vessel 2 including the metal body 3 is filled with the permeation restricting portion 32 shown in FIG. 9, and the permeation restricting portion 32 has a function of restricting the permeation rates of hydrogen and the aqueous solution 5. That is, the permeation restricting portion 32 is the same as the permeation restricting portion 31 except that the volume is different. Accordingly, by filling with the permeation restricting portion 32, the chance of the contact between the metal body 3 and the aqueous solution 5 is reduced, and the reaction rate of corrosion can be decreased. When hydrogen generated by corrosion rises, the rising speed is reduced by the permeation restricting portion 32 having a gel shape. With these actions, the hydrogen generation rate can be controlled.

The permeation restricting portion 32 can cover a plurality of metal bodies 3 simply by filling the lower portion of the culture vessel 2. When the metal body 3 is replenished, the metal body 3 may be charged from above the permeation restricting portion 32. The specific gravity of the metal body 3 is large, and therefore, the metal body 3 can break through the permeation restricting portion 32 and descend to a bottom surface of the culture vessel 2. Therefore, it does not take time and effort to form the permeation restricting portion 31 described above, and the operation is easy.

### 2.5. Fifth Modification

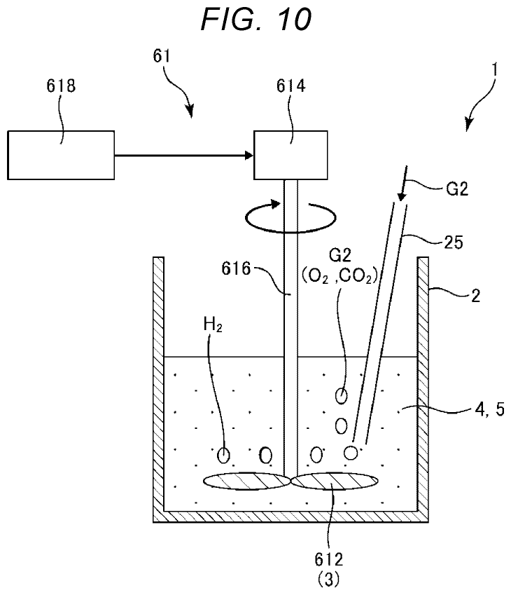

FIG. 10 is a schematic diagram showing the hydrogen-oxidizing bacteria culturing apparatus 1 according to a fifth modification of the embodiment.

Hereinafter, the fifth modification will be described, and in the following description, differences from the embodiment will be mainly described, and description of similar matters will be omitted. In FIG. 10, the same components as those in the embodiment are denoted by the same reference signs.

The hydrogen-oxidizing bacteria culturing apparatus 1 shown in FIG. 10 is the same as the hydrogen-oxidizing bacteria culturing apparatus 1 shown in FIG. 4 except that the metal body 3 is moved relative to the aqueous solution 5.

The hydrogen-oxidizing bacteria culturing apparatus 1 shown in FIG. 10 includes a stirrer 61. The stirrer 61 includes stirring blades 612, a drive unit 614, a shaft 616, and a control unit 618. The stirring blades 612 stir the aqueous solution 5 and the medium 4 by rotation. The stirring blades 612 are implemented by the metal body 3. When the stirring blades 612 rotate, the metal body 3 moves relative to the aqueous solution 5. Accordingly, the aqueous solution 5 having a low dissolved hydrogen concentration normally comes into contact with the metal body 3. As a result, an amount of hydrogen generated per unit time can be increased. The drive unit 614 generates a drive force for rotating the stirring blades 612. The shaft 616 transmits the drive force generated by the drive unit 614 to the stirring blades 612. The control unit 618 changes a rotational speed of the stirring blades 612 by controlling an operation of the drive unit 614. Therefore, the stirrer 61 functions as a moving speed changing unit that freely changes a moving speed of the metal body 3 relative to the aqueous solution 5, for example, from zero to a predetermined value. Accordingly, the amount of hydrogen generated can be adjusted to a target value. The configuration of the moving speed changing unit is not limited to the above-described configuration.

### 2.6. Sixth Modification

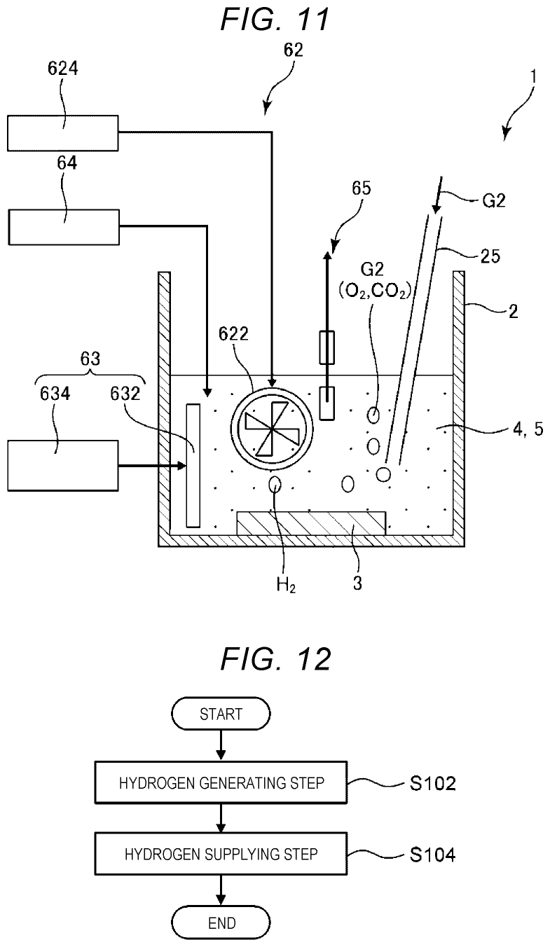

FIG. 11 is a schematic diagram showing the hydrogen-oxidizing bacteria culturing apparatus 1 according to a sixth modification of the embodiment.

Hereinafter, the sixth modification will be described, and in the following description, differences from the embodiment will be mainly described, and description of similar matters will be omitted. In FIG. 11, the same components as those in the embodiment are denoted by the same reference signs.

The hydrogen-oxidizing bacteria culturing apparatus 1 shown in FIG. 11 is the same as the hydrogen-oxidizing bacteria culturing apparatus 1 shown in FIG. 4 except that the aqueous solution 5 flows.

The hydrogen-oxidizing bacteria culturing apparatus 1 shown in FIG. 11 includes a water flow generator 62. The water flow generator 62 includes a pump 622 and a control unit 624. The pump 622 applies a flow rate to the aqueous solution 5. The aqueous solution 5 to which the flow rate is applied moves relative to the metal body 3. Accordingly, as described above, the aqueous solution 5 having a low dissolved hydrogen concentration comes into contact with the metal body 3, and an effective amount of hydrogen generated per unit time can be increased. The control unit 624 changes the flow rate of the aqueous solution 5 by controlling an operation of the pump 622. Therefore, the water flow generator 62 functions as a flow rate changing unit that freely changes the flow rate of the aqueous solution 5, for example, from zero to a predetermined value. Accordingly, the amount of hydrogen generated can be adjusted to a target value. The configuration of the flow rate changing unit is not limited to the above-described configuration.

The hydrogen-oxidizing bacteria culturing apparatus 1 shown in FIG. 11 further includes a temperature regulator 63. The temperature regulator 63 includes a heater 632 and a control unit 634. The heater 632 raises the temperature of the aqueous solution 5. When the temperature of the aqueous solution 5 rises, the metal body 3 is likely to be corroded. Accordingly, the amount of hydrogen generated per unit time can be increased. The control unit 634 changes the temperature of the aqueous solution 5 by controlling an operation of the heater 632. Therefore, the temperature regulator 63 functions as a temperature changing unit that freely changes the temperature of the aqueous solution 5, for example, from room temperature to a predetermined value. Accordingly, the amount of hydrogen generated can be adjusted to a target value. The configuration of the temperature changing unit is not limited to the above-described configuration.

The hydrogen-oxidizing bacteria culturing apparatus 1 shown in FIG. 11 further includes a pH regulator 64 and a sensor 65. The pH regulator 64 controls the pH of the aqueous solution 5. The metal body 3 is likely to be corroded by controlling the pH. Accordingly, the amount of hydrogen generated per unit time can be increased. The sensor 65 feeds back measurement results of the temperature, pH, dissolved hydrogen, dissolved oxygen, and the like of the aqueous solution 5, or measurement results of gas components and the like in the exhaust gas from the aqueous solution 5. Accordingly, the water flow, temperature, and pH are controlled.

### 3. Hydrogen-Oxidizing Bacteria Culturing Method

Next, a hydrogen-oxidizing bacteria culturing method according to the embodiment will be described. In the following description, a method using the hydrogen-oxidizing bacteria culturing apparatus 1 will be described as an example, and an apparatus used in the present culture method is not limited thereto.

FIG. 12 is a flowchart illustrating the hydrogen-oxidizing bacteria culturing method according to the embodiment.

The hydrogen-oxidizing bacteria culturing method shown in FIG. 12 includes a hydrogen generating step S102 and a hydrogen supplying step S104. Hereinafter, the steps will be sequentially described.

### 3.1. Hydrogen Generating Step

In the hydrogen generating step S102, the metal body 3 is brought into contact with the aqueous solution 5 (a liquid containing water). Accordingly, a corrosion reaction is caused in the metal body 3 to generate hydrogen.

For the contact between the metal body 3 and the aqueous solution 5, for example, a method in which the aqueous solution 5 is accommodated in the culture vessel 2 as shown in FIG. 1, and the metal body 3 is charged therein is used.

The metal body 3 preferably contains a metal element having an ionization tendency higher than that of hydrogen. Accordingly, the metal body 3 can efficiently generate hydrogen by the contact with the aqueous solution 5.

The above-described metal element is particularly preferably Ca, Mg, Al, Ti, or Zn. The metal body 3 containing these metal elements has good handleability and hydrogen generation efficiency.

The metal body 3 particularly preferably contains a magnesium-based alloy or a composite material containing a magnesium-based alloy. They are useful as the material of the metal body 3 because the hydrogen generation efficiency is particularly high.

In the hydrogen generating step S102, an operation of adjusting the amount of hydrogen generated from the metal body 3 per unit time may be performed. Examples of such an operation include an operation of changing at least one of the temperature and pH of the aqueous solution 5 (liquid), the flow rate of the aqueous solution 5, and the moving speed of the metal body 3 relative to the aqueous solution 5. The amount of hydrogen generated can be adjusted to a target value by including such an operation. Accordingly, the amount of hydrogen to be wasted can be reduced, and the corrosion of the metal body 3 can be maintained over a longer period of time. As a result, the consumption efficiency of the metal body 3 can be increased.

In the hydrogen generating step S102, the amount of hydrogen may be adjusted by adding a member for the metal body 3. For example, as shown in FIGS. 6, 7, 8, and 9, the metal body 3 may be brought into contact with the aqueous solution 5 (liquid) in a state of being respectively covered with the permeation restricting portions 32, 31, 34, and 36. The permeation restricting portions 31, 32, 34, and 36 have a function of restricting the permeation rates of the aqueous solution 5 and hydrogen. The amount of hydrogen generated can be adjusted to a target value by adding such a member. Accordingly, the consumption efficiency of the metal body 3 can be increased.

### 3.2. Hydrogen Supplying Step

In the hydrogen supplying step S104, the hydrogen generated in the hydrogen generating step S102 is supplied to the medium 4 inoculated with hydrogen-oxidizing bacteria. Accordingly, the hydrogen-oxidizing bacteria grow to implement carbon dioxide fixation. As a result, various chemical products are produced.

The hydrogen supplying step S104 may include an operation of supplying carbon dioxide or oxygen and carbon dioxide to the medium 4 as shown in FIG. 1 and the like. Carbon dioxide or oxygen and carbon dioxide can be continuously supplied to the medium 4 by including such an operation. As a result, the hydrogen-oxidizing bacteria can be continuously cultured, and the production of chemical products based on carbon dioxide fixation can be continuously performed.

The hydrogen generating step S102 and the hydrogen supplying step S104 may be performed simultaneously. That is, the hydrogen generating step S102 and the hydrogen supplying step S104 may not be distinguished from each other in terms of time. Accordingly, the time and effort for managing the step can be saved, and the labor can be saved. In order to simultaneously perform both steps, for example, the culture vessel 2 shown in FIG. 4 and the like may be used.

As described above, the hydrogen-oxidizing bacteria can be cultured. The cultured hydrogen-oxidizing bacteria contain the produced chemical products, and therefore, the produced chemical products may be collected by any recovery method. Examples of the collection method include various separation methods such as fractionation, extraction, ultrasonic atomization separation, chromatography, and crystallization. The chemical products to be produced are not particularly limited, and examples thereof include ethanol, isobutanol, and lactic acid. The cultured hydrogen-oxidizing bacteria themselves can be used as chemical products. Specifically, the cultured hydrogen-oxidizing bacteria can be used as various feeds such as livestock feeds and fish culture feeds, protein resources, and the like.

### 4. Effects of Embodiment

As described above, the hydrogen-oxidizing bacteria culturing method according to the embodiment includes the hydrogen generating step S102 and the hydrogen supplying step S104. In the hydrogen generating step S102, hydrogen is generated by bringing the metal body 3 into contact with the aqueous solution 5 (a liquid containing water) and causing a corrosion reaction in the metal body 3. In the hydrogen supplying step S104, the generated hydrogen is supplied to the medium 4 inoculated with hydrogen-oxidizing bacteria.

According to such a hydrogen-oxidizing bacteria culturing method, hydrogen can be generated at a necessary timing without storing or transporting a large amount of hydrogen. Compression or liquefaction of hydrogen at a high pressure is not involved in the supply of hydrogen, and therefore, energy consumption in the supply of hydrogen can be reduced. Accordingly, the hydrogen-oxidizing bacteria can be efficiently cultured at low cost while reducing equipment investment. As a result, the carbon dioxide fixation is implemented, so that a chemical product can be produced while contributing to carbon neutrality.

The metal body 3 preferably contains a metal element having an ionization tendency higher than that of hydrogen. Accordingly, the metal body 3 can efficiently generate hydrogen by contact with the aqueous solution 5.

The above-described metal element is preferably Ca, Mg, Al, Ti, or Zn. The metal body 3 containing these metal elements has good handleability and hydrogen generation efficiency.

The metal body 3 preferably contains a magnesium-based alloy or a composite material containing a magnesium-based alloy. They are useful as a material of the metal body 3 because the hydrogen generation efficiency is particularly high.

The hydrogen supplying step S104 may include an operation of supplying carbon dioxide or oxygen and carbon dioxide to the medium 4. Carbon dioxide or oxygen and carbon dioxide can be continuously supplied to the medium 4 by including such an operation. As a result, the hydrogen-oxidizing bacteria can be continuously cultured, and production of chemical products based on carbon dioxide fixation can be continuously performed.

The hydrogen generating step S102 may include an operation of changing at least one of the temperature and pH of the aqueous solution 5 (liquid), the flow rate of the aqueous solution 5, and the moving speed of the metal body 3 relative to the aqueous solution 5. The amount of hydrogen generated can be adjusted to a target value by including such an operation. Accordingly, the amount of hydrogen to be wasted can be reduced, and the corrosion of the metal body 3 can be maintained over a longer period of time. As a result, the consumption efficiency of the metal body 3 can be increased.

The hydrogen generating step S102 may include an operation of bringing the metal body 3 into contact with the aqueous solution 5 (liquid) in a state where the metal body 3 is separately covered with the permeation restricting portion 31, 32, 34, or 36 that restricts the permeation rates of the aqueous solution 5 (liquid) and hydrogen. The permeation restricting portions 31, 32, 34, and 36 have a function of restricting the permeation rates of the aqueous solution 5 and hydrogen. The amount of hydrogen generated can be adjusted to a target value by including such an operation. Accordingly, the consumption efficiency of the metal body 3 can be increased.

The hydrogen-oxidizing bacteria culturing apparatus 1 according to the embodiment includes the culture vessel 2, the metal body 3, and the medium 4. The metal body 3 is accommodated in the culture vessel 2, and contact between the metal body 3 and the aqueous solution 5 (a liquid containing water) causes a corrosion reaction to generate hydrogen. The medium 4 is accommodated in the culture vessel 2 in a manner of coming into contact with the generated hydrogen, and is inoculated with hydrogen-oxidizing bacteria.

According to such a hydrogen-oxidizing bacteria culturing apparatus 1, hydrogen can be generated at a necessary timing without storing or transporting a large amount of hydrogen. Compression or liquefaction of hydrogen at a high pressure is not involved in the supply of hydrogen, and therefore, energy consumption in the supply of hydrogen can be reduced. Accordingly, the hydrogen-oxidizing bacteria can be efficiently cultured with even an inexpensive culturing apparatus having a simple structure. As a result, the carbon dioxide fixation is implemented, so that a chemical product can be produced while contributing to carbon neutrality.

As shown in FIG. 4 and the like, the culture vessel 2 may accommodate the aqueous solution 5 (liquid) and the medium 4 in a mixed state. In such a culture vessel 2, the aqueous solution 5 and the culture medium 4 are mixed, and therefore, the time and effort for supplying the generated hydrogen can be saved. Accordingly, the structure of the culture vessel 2 can be simplified, and consumption of energy necessary for supplying hydrogen can be reduced.

The culture vessel 2 may include the first accommodating unit 21, the second accommodating unit 22, and the hydrogen transfer unit 23. The first accommodating unit 21 accommodates the metal body 3 and the aqueous solution 5 (liquid). The second accommodating unit 22 accommodates the medium 4. The hydrogen transfer unit 23 transfers hydrogen generated in the first accommodating unit 21 to the second accommodating unit 22.

According to such a culture vessel 2, the aqueous solution 5 and the medium 4 can be stored separately. Therefore, for example, the influence of the components in the aqueous solution 5 on the growth of the hydrogen-oxidizing bacteria can be avoided.

The hydrogen transfer unit 23 may include a hydrogen permeable film. The hydrogen permeable film selectively allows hydrogen generated in the first accommodating unit 21 to permeate through the second accommodating unit 22. Accordingly, it is possible to prevent the transfer of oxygen dissolved in the aqueous solution 5, a by-product accompanying the generation of hydrogen, and the like to the medium 4. As a result, it is possible to prevent inhibition of growth of the hydrogen-oxidizing bacteria due to transfer of components other than hydrogen.

The hydrogen transfer unit 23 may transfer the hydrogen generated in the first accommodating unit 21, together with the aqueous solution 5 (liquid), to the second accommodating unit 22. Accordingly, the aqueous solution 5 containing dissolved hydrogen instead of hydrogen as a gas can be transferred, and therefore, a high dissolved hydrogen concentration can be efficiently transferred. As a result, the hydrogen-oxidizing bacteria can be efficiently cultured in the medium 4.

The hydrogen-oxidizing bacteria culturing apparatus 1 may include the gas supply unit 25. The gas supply unit 25 supplies carbon dioxide or oxygen and carbon dioxide to the medium 4. Accordingly, carbon dioxide or oxygen and carbon dioxide can be continuously supplied to the medium 4. As a result, the hydrogen-oxidizing bacteria can be continuously cultured.

The hydrogen-oxidizing bacteria culturing apparatus 1 includes at least one of the temperature regulator 63 as an example of the temperature changing unit that changes the temperature of the aqueous solution 5 (liquid), the pH regulator 64 as an example of the pH changing unit that changes the pH of the liquid, the water flow generator 62 as an example of the flow rate changing unit that changes the flow rate of the aqueous solution 5, and the stirrer 61 as an example of the moving speed changing unit that changes the moving speed of the metal body 3 relative to the aqueous solution 5. Accordingly, the amount of hydrogen generated can be adjusted to a target value. As a result, the consumption efficiency of the metal body 3 can be increased.

Although the hydrogen-oxidizing bacteria culturing method and the hydrogen-oxidizing bacteria culturing apparatus according to the present disclosure have been described above based on the shown embodiment, the present disclosure is not limited to the embodiment. For example, the hydrogen-oxidizing bacteria culturing method according to the present disclosure may be a method in which any step is added to the embodiment. The hydrogen-oxidizing bacteria culturing apparatus according to the present disclosure may be an apparatus in which any component is added to the embodiment.

What is claimed is:

1. A hydrogen-oxidizing bacteria culturing method comprising:
a hydrogen generating step of generating hydrogen by bringing a metal body into contact with a liquid containing water and causing a corrosion reaction in the metal body; and
a hydrogen supplying step of supplying the generated hydrogen to a medium inoculated with hydrogen-oxidizing bacteria.

2. The hydrogen-oxidizing bacteria culturing method according to claim 1, wherein
the metal body contains a metal element having an ionization tendency higher than an ionization tendency of hydrogen.

3. The hydrogen-oxidizing bacteria culturing method according to claim 2, wherein
the metal element is Ca, Mg, Al, Ti, or Zn.

4. The hydrogen-oxidizing bacteria culturing method according to claim 3, wherein
the metal body contains a magnesium-based alloy or a composite material containing a magnesium-based alloy.

5. The hydrogen-oxidizing bacteria culturing method according to claim 1, wherein
the hydrogen supplying step includes an operation of supplying carbon dioxide or oxygen and carbon dioxide to the medium.

6. The hydrogen-oxidizing bacteria culturing method according to claim 1, wherein
the hydrogen generating step includes an operation of changing at least one of a temperature and pH of the liquid, a flow rate of the liquid, and a moving speed of the metal body relative to the liquid.

7. The hydrogen-oxidizing bacteria culturing method according to claim 1, wherein
the hydrogen generating step includes an operation of bringing the metal body into contact with the liquid in a state where the metal body is covered with a permeation restricting portion configured to restrict permeation rates of the liquid and hydrogen.

8. A hydrogen-oxidizing bacteria culturing apparatus comprising:
a culture vessel;
a metal body accommodated in the culture vessel and configured to generate hydrogen by causing a corrosion reaction by contact with a liquid containing water; and
a medium accommodated in the culture vessel so as to be in contact with the generated hydrogen, and inoculated with hydrogen-oxidizing bacteria.

9. The hydrogen-oxidizing bacteria culturing apparatus according to claim 8, wherein
the culture vessel accommodates the liquid and the medium in a mixed state.

10. The hydrogen-oxidizing bacteria culturing apparatus according to claim 8, wherein
the culture vessel includes:
a first accommodating unit configured to accommodate the metal body and the liquid;
a second accommodating unit configured to accommodate the medium; and
a hydrogen transfer unit configured to transfer the hydrogen generated in the first accommodating unit to the second accommodating unit.

11. The hydrogen-oxidizing bacteria culturing apparatus according to claim 10, wherein
the hydrogen transfer unit transfers the hydrogen generated in the first accommodating unit, together with the liquid, to the second accommodating unit.

12. The hydrogen-oxidizing bacteria culturing apparatus according to claim 8, further comprising:
a gas supply unit configured to supply carbon dioxide or oxygen and carbon dioxide to the medium.

13. The hydrogen-oxidizing bacteria culturing apparatus according to claim 8, further comprising:
at least one of a temperature changing unit configured to change a temperature of the liquid, a pH changing unit configured to change pH of the liquid, a flow rate changing unit configured to change a flow rate of the liquid, and a moving speed changing unit configured to change a moving speed of the metal body relative to the liquid.
